(19) United States
(12) Patent Application Publication
(10) Pub. No.: US 2003/0124148 A1
(43) Pub. Date: Jul. 3, 2003

(54) Method for producing autogenous vaccines for treating chlamydial infections in mammals and humans

(76) Inventors: Hans-Guenther Sonntag, Bammental (DE); Oliver Nolte, Eppelheim (DE); Hannelore Weiss, Neckargemuend (DE); Hans-Erich Weiss, Nackargemuend (DE)

Correspondence Address: FOLEY AND LARDNER, SUITE 500, 3000 K STREET NW, WASHINGTON, DC, 20007, US

(21) Appl. No.: 09/869,436

(22) PCT Filed: Dec. 16, 1999
(86) PCT No.: PCT/EP99/09993

(30) Foreign Application Priority Data
Dec. 28, 1998 (EP) 198 60 438.6

(51) Int. Cl. A61K 39/118

(52) U.S. Cl. 424/263.1

(57) ABSTRACT

The invention relates to a method for treating chlamydial infections in mammals and humans. According to the invention, tissue material containing clamydia is heated to temperatures of greater than 50° C., preferably 80-85° C., for 1-5 hours in the presence of agents, preferably formaldehyde or p-formaldehyde, which cross-link proteins.

The present invention concerns a novel process for the preparation of autovaccines for the treatment of chlamydioses of mammals and humans.

Chlamydia are intracellular parasitising pathogenic micro-organisms. They multiply only in living cells, thus e.g. in the tissue of attacked organ systems, such as uterus and lungs. In rejected, destroyed cell layers, such as uterine mucosa, placentas and pleura exudates, they are to be detected on a massive scale (immunofluorsecence).

It is known that chlamydia are only insufficiently combatted by the body's own defence mechanism in the human or animal body and are only poorly accessible to a chemotherapy.

Chlamydia are a frequent cause for genital infections (NGU=non-gonorrhoeal urethritis) and, in the case of sheep and cattle, lead to miscarriages and, in the case of humans, partly to sterility (chlamydia trachomatis). In the case of domestic cats, there occurs a causative agent of the cat scratching disease belonging to the group of chlamydia, whereby keratoconjunctivitides are triggered. Furthermore, there is seen a causal connection between a chlamydia pneumonia infection and cases of coronary heart disease.

From U.S. Pat. No. 4,242,686 is known a cat vaccine in which chlamydia cells originating from a depository are precultured in hens' eggs and kidney cell cultures and further cultured in Eagle's MEM medium before the chlamydia are inactivated by addition of chemicals, such as ethyleneimine, formalin, thimerosal, glutaraldehyde, phenol etc. or by heating to 56° C. for 5 min. or by UV or gamma irradiation and the mixture is brought into a form suitable as vaccine. This vaccine is not suitable for the defence against mutated chlamydia infections.

From U.S. Pat. No. 4,328,208 are known vaccines in which chlamydia cultured in hens' eggs are suspended in phosphate buffer solution and inactivated with glutaraldehyde, merthiolate and/or formaldehyde at 37° C. for 10 hours. To the suspension is added an amine and a polyacrylamide gel and ground in a colloid mill to injection fineness. The mixture is to be used as intramuscular injection for chlamydia prophylaxis in any desired animals.

DD 216385 describes a similar vaccine in which the hens' egg/chlamydia suspension inactivated with ethyleneimine for 20 hours is bound to aluminium hydroxide gel.

WO 93-04163 describes the use of a chlamydia Sp strain isolated from hamsters for the production of vaccines against other chlamydia strains whereby there can be used either corresponding antibodies or the hamster chlamydia themselves not infectious for the patient or samples inactivated by means of formalin. Because of the only partial correlation with the chlamydia to be treated, the action is limited.

From the earlier or still normal therapy of the flocks of sheep infected with chlamydia with tetracycline and its derivatives, it is known that it is not possible to free all animals from chlamydia so that, year for year, especially in the case of primaparous animals, as previously abortions occur at a rate of 20-40% of the cases.

By means of the immunisation according to the invention with an autovaccine from the causative agent attacking the herd, one achieves a reduction of the abortions to less than 3%.

The use of an autovaccine in human gynaecology promises a similar high rate in the prevention of the chlamydia trachomatic-caused sterilities.

Autovaccines from chlamydia were successfully used in the experimental stage for the combatting of the pleuropneumonias in cats based on pleura exudate.

The process according to the invention uses the chlamydia present in the cell and organ layers or exudates in the particular host-specific or own tissues and/or body fluids of the attacked species (cats, sheep, cattle, humans).

The particular material with the intracellular chlamydia is isolated either via rejected organ parts (placenta), shavings of the mucous membrane (uterus)or by puncture of the thorax (pleura exudate).

From the placenta of a lamb or calf, so much tissue attacked with chlamydia can be removed that it is sufficient to inject a sheep or cattle flock of 500 or 100 animals 3-4 times with, in each case, 2.0 or 5.0 ml, respectively.

Therefore, the present invention has set itself the task of making available an autovaccine against chlamydia which acts specifically and is thus able substantially to increase the chances of healing. By autovaccine in the scope of this invention is understood not only a vaccine which has been obtained from the patient's own material but also those vaccines which were obtained from other patients present in comparatively long spatial contact with the patients, for example in several animals of a herd or humans of a shared dwelling or the same working place.

The solution takes place with a process according to the main claim. Advantageous embodiments of this process are found in the subsidiary claims.

The agent according to the invention represents an autovaccine which e.g. in the case of appropriate working up can be administered subcutaneously and/or perorally via the mucous membrane.

The process starts from the chlamydia contained in the cells or organ layers or exudates of the attacked organisms with inclusion of the other components, such as cell membranes and proteins, blood cells, as well as toxoids brought about by the chlamydia etc., in the following designated as tissue material.

By means of the treatment of the tissue material at elevated temperatures in the presence of cross-linking reagents, all proteins contained are individually cross-linked and thereby denatured, i.e. on the chlamydia and other products contained in the tissue material, it is acted upon in a specific way. Surprisingly, this treatment leads to the formation of strongly antigen-acting products which, in the case of one or possibly multiple application to the organisms attacked with chlamydia leads to a protective immune response although the amount of the chlamydia in the so treated and administered tissue material accounts for only a fraction of the amount of chlamydia present in the organism due to the disease. The process according to the invention differs significantly from the preparation of bacterial autovaccines in which first a bacteria sample is multiplied for the provision of large amounts of antigenic material and only thereafter the bacteria isolated, killed and administered as vaccine in comparatively large amount.

Due to the cross-linking and the temperature treatment, the tissue material can be solidifed and there form the particles inducing an anti-causative agent-specific immune response. In order to obtain a liquid consistency suitable for working up and for use, there is thereby added thereto, with stirring, so much pyrogen-free physiological common salt solution as is necessary.

For the production of a vaccine to be administered subcutaneously, the so obtained mixture is filtered over filters with pore sizes of below 400 μm in order to separate off comparatively large complexes. The liquefied agent can also be administered to the patient via the mucous membranes of the mouth/throat (gargling).

For the cross-linking of the proteins, formalin has proved to be especially suitable, this being added to the tissue material in an amount of at least about 0.1 to about 1.0 volume percent in the form of a saturated formalin solution. Paraformaldehyde, glutaraldehyde, PLP (periodate-lysine-paraformaldehyde), carbodiimides, especially 1-ethyl-3-(3-dimethylaminopropyl)-carbodiimide or 3,3′-dimethylsuberimidate are to mentioned as further examples for cross-linking-acting reagents.

The denaturing temperature in the presence of cross-linking reagents lies at above 50° C., preferably between 80 and 85° C., whereby one maintains this temperature for 1-5 hours, preferably about 2 hours. This time suffices in order to subject the chlamydia, as well as chlamydia-containing cells, to a denaturing and cross-linking from which results a change of the antigeneity.

This treatment of the tissue material and of the antigens obtained (causative agent) lead to a change of the antigenic properties by denaturing of the proteins (heat) in the case of simultaneous cross-linking (formalin or formaldehyde or paraformaldehyde or phenol or its derivatives). This denaturing plus cross-linking has the result that the antigen (the causative agent) is accessible to the immune system in a way other than the native one. It results therefrom that also causative agents which possibly remain unrecognised in the native state or induce a non-adequate form of the immune response (chronic inflammatory course or the like) can be combatted. It is assumed that, besides the causative agents themselves, other components/receptors of the cells, body inherent or formed by the infection, also cross-link and are prepared for the contact with the antibody-forming cells or the immune system and are made available and that thereby the increased activity of the autovaccines according to the invention is brought about.

These considerations were supported by extensive investigations not only with animal but also, in part, with human patients. To the greater part, it was thereby a question of chronically persisting or recidivising infections. Thus, one can start from the point that the antigen was fundamentally accessible to the immune system. After administration of the antigen in denatured and cross-linked form, in most cases in less than four weeks it came to a healing of the infection. This can only be explained by the described altered form of presentation of the antigen by heat and formaldehyde.

### EXAMPLE I

From the fresh placenta of an aborted lamb or calf infected with chlamydia are removed 300 or 500 g, respectively of tissue material and first heated about 60 min. at 95° C. Subsequently, the placenta parts are comminuted as far as possible with a comminution apparatus and thereafter passed through a sterile sieve (pore width 400 μm) or over a sterile funnel into a sterile flask.

This mass is diluted about 1:3 with sterile, pyrogen-free physiological NaCl solution, mixed with 0.3 vol. % formalin and again heated for 2 hrs. at 80° C., incubated for 24 hrs. at 37° C. and subsequently tested for sterility.

In the case of negative sterility test, the autovaccine is ready for use.

The vaccine obtained is sufficient in order to inject 3-4 times with, in each case, 2.0 or 5.0 ml, respectively, a flock of sheep or cattle, respectively, of 500 or 1000 animals, respectively, infected with chlamydia. The so treated mother animals show an abortion rate of below 3% compared with untreated animals which show an abortion rate of 20-40%.

### EXAMPLE II

For the treatment of chlamydia (psittaci, pneumoniae or trachomatis) infections in humans, there is taken either a pleura exudate or, in the case of women, a mucous membrane smear from the uterus. In the latter case, samples of several smears are collected and temporarily stored at −20° C. until the necessary total amount of 5-6 ml is achieved in order to suffice for the immunisation of the patient. This sample is comminuted and treated analogously to Example I.

In the case of availability of appropriate amounts of material, chlamydia pneumoniae autovaccines can be used for the influencing of inflammatory process of the coronary liquid caused by pneumoniae.

The administration in the case of humans takes place subcutaneously under the abdominal skin in 4 injections of 1.5 to 3 ml. If sufficient vaccine mass (pleura exudate) is available, it is recommended to carry out parallel thereto a peroral administration of 5-10 ml/day over the first 10 days.

Shock situations are not to be expected since the protein surrounding the chlamydia is species-specific. Nevertheless, in the case of humans, an allergy test is recommended before the first s.c. administration.

1. Process for the preparation of autovaccines for the treatment of chlamydioses of mammals and humans, characterised in that one heats chlamydia-containing tissue material, without propagation step for the chlamydia, in the presence of cross-linking agents to temperatures above 50° C. to 90° C. and brings to an administerable liquid consistency.

2. Process according to claim 1, characterised in that, as protein cross-linking agent, one adds formalin, paraformaldehyde, glutaraldehyde, PLP (periodatelysine-paraformaldehyde), carbodiimides, especially 1-ethyl-3-(3-dimethylaminopropyl)-carbodiimide or 3,3′-dimethylsuberimidate and maintains the temperature for 2 hours at 80-85° C.

3. Process according to claim 1 or 2, characterised in that one adds pyrogen-free physiological common salt solution for the liquefaction.

4. Process according to one of claims 1 to 3, characterised in that and passes the vaccines through a filter with a pore width of below 400 μm.

5. Process according to one of claims 1 to 4, characterised in that to the tissue material are added 0.1 to 1.0, preferably 0.5 volume percent of a saturated formalin solution.

6. Autovaccines for the treatment of chlamydioses of mammals and humans containing antigens produced by denaturing according to claim 1 to 5.
